(19) United States
(12) Patent Application Publication
(10) Pub. No.: US 2003/0211058 A1
(43) Pub. Date: Nov. 13, 2003

(54) Skin care compositions

(76) Inventors: Paul Jonathan Matts, Surrey (GB); Paul Elliott Mann, Hertfordshire (GB); Jonathan David Webster, Surrey (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/181,653

(22) PCT Filed: Jan. 18, 2001
(86) PCT No.: PCT/US01/01827

(30) Foreign Application Priority Data
Jan. 19, 2000 (GB) 0001235.1
Jan. 20, 2000 (GB) 0001311.0

(51) Int. Cl. A61K 7/021

(52) U.S. Cl. 424/63

(57) ABSTRACT

The present invention relates to a topical, liquid, cosmetic composition for effecting immediate improvements in the appearance and feel of skin, in particular for providing a more even skin tone. The composition comprises: a) from 0.3% to 1.5% of an optically neutral phyllosilicate; and b) a green pigment in an amount effective to give the composition a Δa colour shift value of from −1 to −5 and a Δb colour shift value of from −2 to −5, when applied to the skin or a skin substitute at a coverage of 2.4 mg/cm^{-2.}, wherein the total level of inorganic particles in the composition is less than 5%. In a second aspect of the invention, the composition comprises: a) from 0.3% to 1.5% of an optically neutral phyllosilicate; and b) a green platelet-type interference pigment, having a TiO₂ layer thickness of from 120 nm to 160 nm or a whole number multiple thereof, in an amount of from 0.1% to 0.5% by weight of the composition; wherein the total level of inorganic particles in the composition is less than 5%. The compositions are preferably oil in water emulsions.

### TECHNICAL FIELD

The present invention relates to the field of topical compositions for improving the appearance and feel of human skin. More particularly, the invention relates to topical compositions which provide good coverage of skin imperfections, e.g., pores and uneven skin tone, while retaining a natural skin appearance. The compositions of the invention are especially useful for the cosmetic treatment of non-facial parts of the body, especially the hands.

### BACKGROUND

Topical cosmetic compositions, such as skin creams and lotions and the like, can be designed to deliver a variety of post-application benefits to the skin, such as moisturisation, skin protection and improvements to skin feel and appearance. Such benefits may be entirely cosmetic or may be delivered by the use of chronically acting actives which are capable of regulating skin condition, including regulating fine lines, wrinkles and other forms of uneven or rough surface texture associated with aged or photodamaged skin. Ideally, skin products will not only deliver benefits on a longer time scale but will also effect immediately noticeable improvements such as enhanced skin smoothness. For commercial success it is further desirable that products have excellent in-use aesthetics i.e. that they can readily be rubbed in without feeling greasy or sticky.

It is known to use green coloured pigments to offset skin redness. For example, WO 96/03962 describes make-up compositions which comprise TiO₂ coated mica interference pigments for that purpose and U.S. Pat. No. 5,690,916 describes methods of making hyperchromic portions of the skin (spots, freckles and the like) inconspicuous by using similar materials.

Whilst the compositions and disclosures of the prior art provide useful advances in the art of cosmetic skin treatment, there remains the need for improved compositions which deliver immediate improvements in skin feel and appearance, particularly for non-facial parts of the body, and especially for environmentally stressed areas of the body such as hands. The compositions also need to be non-greasy and easy to apply.

In particular, it has now been found that enhanced skin feel and appearance for a large variety of skin colours can be obtained by employing controlled amounts of both optically neutral and coloured platelet type particles in liquid, topical compositions, which are preferably oil-in-water emulsions. Prior art compositions comprising larger amounts of interference pigments, or absent optically neutral platelet particles, have been found not to give the same degree of colour control. The skin smoothness effects and in-use aesthetics of the compositions of the invention are particularly enhanced when the compositions comprise an oil phase which has a degree of spreadability and lubricity suited to the platelet particle system.

The present invention also relates to cosmetic methods of improving skin feel and appearance, in particular for providing a more even skin tone, by topical application of the subject compositions.

### SUMMARY OF THE INVENTION

In a first aspect, the present invention relates to a topical, liquid, cosmetic composition, preferably in the form of an oil in water emulsion, comprising:

a) from about 0.3% to about 1.5% of an optically neutral phyllosilicate; and

b) a green pigment in an amount effective to give the composition a Δa colour shift value of from −1 to −5 and a Δb colour shift value of from −2 to −5, when applied to the skin or a skin substitute at a coverage of 2.4 mg cm^{-2.},

wherein the total level of inorganic particles in the composition is less than 5%.

In a second aspect, the present invention relates to a topical, liquid, cosmetic composition, preferably in the form of an oil in water emulsion, comprising:

a) from about 0.3% to about 1.5% of an optically neutral phyllosilicate; and

b) a green platelet-type interference pigment, having a TiO₂ layer thickness of from 120 nm to 160 nm or a whole number multiple thereof, in an amount of from 0.1% to 0.5% by weight of the composition;

wherein the total level of inorganic particles in the composition is less than 5%.

The compositions are useful for imparting an essentially immediate visual improvement in skin appearance and feel while maintaining a natural skin appearance.

The invention further relates to cosmetic methods of improving skin appearance and feel by topical application of the subject compositions.

### DETAILED DESCRIPTION OF THE INVENTION

All percentages and ratios used herein are by weight of the total composition and all measurements made are at 25° C., unless otherwise designated. Contact angles cited are advancing contact angles on hydrated collagen sheet.

All publications cited herein are hereby incorporated by reference in their entirety.

By “topical, liquid, cosmetic composition” is meant a wet or oily composition which is intended to be rubbed into the skin. Preferred compositions are in the form of a lotion, cream or a gel.

The term “dermatologically-acceptable” as used herein, means that the compositions or components thereof so described are suitable for use in contact with human skin without undue toxicity, incompatibility, instability, allergic response, and the like.

The term “safe and effective amount” as used herein means an amount of a compound, component, or composition sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound medical judgement.

The term “optically neutral” means that when a material is applied to the skin by itself it effects no net colour change other than a possible lightening or darkening of tone. Preferably the Δa and Δb colour shift values defined herein in the section headed ‘Green Pigments’ will each have a magnitude no greater than one unit, more preferably no greater than one half of one unit, for an optically neutral material. Such materials will normally be colourless, white or off-white in bulk appearance.

Active and other ingredients useful herein may be categorised or described herein by their cosmetic and/or therapeutic benefit or their postulated mode of action. However, it is to be understood that the active and other ingredients useful herein can in some instances provide more than one cosmetic and/or therapeutic benefit or operate via more than one mode of action. Therefore, classifications herein are made for the sake of convenience and are not intended to limit an ingredient to the particularly stated application or applications listed.

The topical compositions useful in the subject invention may be made into a variety of product forms such as are known in the art. Preferred compositions have a sufficient thickness or yield point to resist sedimentation of the particulates.

Preferred compositions contain a dermatologically acceptable, hydrophilic diluent. Suitable hydrophilic diluents include water, organic hydrophilic diluents such as C₁-C₄ monohydric alcohols and low molecular weight glycols and polyols, including propylene glycol, polyethylene glycol (e.g. of MW 200-600), polypropylene glycol (e.g. of MW 425-2025), glycerol, butylene glycol, 1,2,4-butanetriol, sorbitol esters, 1,2,6-hexanetriol, ethanol, iso-propanol, sorbitol esters, ethoxylated ethers, propoxylated ethers and combinations thereof. The diluent is preferably liquid. Water is an especially preferred diluent. The composition preferably comprises at least about 60% of the hydrophilic diluent.

Highly preferred carriers comprise an emulsion comprising a hydrophilic phase, especially an aqueous phase, and a hydrophobic phase e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 99% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. Highly preferred compositions herein are oil-in-water emulsions.

Preferred compositions have an apparent viscosity of from about 5,000 to about 200,000 mPa.s (centipoise). For example, preferred lotions have an apparent viscosity of from about 10,000 to about 40,000 mPa.s; preferred creams have an apparent viscosity of from about 30,000 to about 160,000 mPa.s. Apparent viscosity can be determined using a Brooldield DVII RV viscometer, spindle TD, at 5 rpm, or the equivalent thereof. The viscosity is determined on the composition after the composition has been allowed to stabilise following its preparation, generally at least 24 hours under conditions of 25° C.±1° C. and ambient pressure after preparation of the composition. Apparent viscosity is measured with the composition at a temperature of 25° C.±1° C., after 30 seconds spindle rotation.

The compositions of the present invention are usually formulated to have a pH of 8.5 or below and in general have a pH in the range from about 4.5 to about 7.5, more preferably from about 5 to about 6.5. Some compositions, particularly those comprising an additional active such as salicylic acid, require a lower pH in order for the additional active to be fully efficacious. These compositions are usually formulated to have a pH of from about 2.5 to about 5, more preferably from about 2.7 to about 4.

Optically Neutral Phyllosilicates

An essential component of the compositions of the present invention is an optically neutral phyllosilicate, which both enhances skin feel and contributes to the net colour change effected by the compositions. The phyllosilicates are a subclass of silicates in which the atoms are arranged in weakly bonded sheets such that they create lamellar particles on grinding. Phyllosilicates as a class incorporate numerous subclasses, described for example on the Internet at http://mineral.galleries.com/minerals/silicate/phyllosi.htm. Many individual subclasses are also described in the Kirk-Othmer Encyclopaedia of Chemical Technology 4^{th} Edn. Preferred subclasses include kaolonite, montmorillonite, talc and mica. Most preferred are talc and mica, especially mica. The layer structure of the phyllosilicates gives a smooth feeling upon application to the skin. The most preferred materials from the point of view of appearance and skin feel are optically neutral titanium coated micas. These optically neutral titanium coated micas are commercially available and suitable materials include Microna Matte White, available from Rona, a subsidiary of Merck.

The optically neutral phyllosilicate is generally present at a level of from about 0.3% to about 1.5%, preferably from about 0.5% to about 1.3%, more preferably from about 0.6% to about 1.0%. Lower levels do not give the skin feel or colour adjustment benefits of the present invention whilst higher levels can give a heavy feeling or appearance.

Overall particle sizes for the optically neutral phyllosilicates are preferably within the range from about 0.1 to about 100 μm, more preferably from about 0.5 to about 20 μm for the largest dimension and preferably from about 100 to about 1500 nm in thickness.

Green Pigments

A further essential component of the compositions of the first aspect of the present invention is a green pigment in an amount effective to give the composition a Δa colour shift value of from −1 to −5, preferably from −2 to −4 and a Δb colour shift value of from −2 to −5, preferably from −2 to −4, more preferably from −2 to −3 when applied to the skin or a skin substitute at a coverage of 2.4 mg cm². The test methodology for measuring colour shift values is described in detail below. It has been found that colour shifts within the ranges claimed are effective to reduce skin redness without creating an unnatural greenness on darker skin tones. In this way the green pigment assists in providing an overall even skin tone. Similar Δa colour shifts without an accompanying Δb colour shift can be acceptable on normally pale skin, such as Caucasian skin tones but it has been found that it creates an excessive perception of greenness on darker skin tones. The use of an optically neutral phyllosilicate, as herein described, together with the green pigment allows the desired colour shifts which are acceptable for a wide range of skin types.

Whilst the desired colour shifts are of course those for human skin, it has been found that these can be closely approximated by an in-vitro test. Accordingly claimed colour shifts refer to those measured on the following in vitro test.

A flat piece of absorbent, translucent surgical glove is laid (absorbent surface uppermost) onto a standard red tile and the colour of the whole system is measured using a suitable reflectance spectrophotometer (e.g. the Minolta CM-512M3), illuminating with D65 light at an angle of 25° to the normal and measuring at an angle of 10° to the normal. The glove-on-tile set-up has been found to mimic the behaviour and colour of human skin sufficiently well for the purpose of the test. The tile used should be the “Red” chip from a standard colour chart (Macbeth Color Checker) as supplied by Macbeth Munsell Colour, New York, USA. The type of glove is not especially critical. The type used by the present applicants, however, is a Biogel surgical glove as supplied by Regent Hospital Products, Broxboume, UK. The measured colour of the glove laid on the tile is used to generate a set of reference L, a, b value. The composition whose colour shift is to be measured is then applied to the glove at a coverage of 2.4 mg cm^{-2,} using a pre-saturated finger cot and is rubbed in for 30 seconds. The product is then allowed to dry for 10 minutes and the colour is re-measured in the same way as before to generate a set of post-treatment L, a, b values. The L, a, b values of the reference are subtracted from the post-treatment L, a, b values to generate ΔL, Δa and Δb values for the composition. These values being used to characterise completely the colour shift. The test numbers should be averaged over ten different repeats.

Suitable green pigments can include interference pigments and reflectance pigments. Suitable reflectance pigments include chromium hydroxide, such as the materials available under the tradenames Chroma Lite Aqua 4508 from ISP Van Dyck and H Chrome Green 105 from Presperse, and chromium oxide, such as Chroma Lite Green from ISP Van Dyck and Gemtone Emerald from Mearl. Preferably the green pigment is a platelet-type interference pigment material having a TiO₂ layer thickness of from about 120 nm to about 160 nm or a whole number multiple thereof. Preferably, the interference pigment material comprises platelet type mica which is coated with TiO₂. The colour of the reflected light varies depending on the thickness of the layer. The interference pigment material used in the present invention comprises at least a proportion of pigment material having a TiO₂ layer thickness of from about 120 nm to about 160 nm or a whole number multiple thereof such that the pigment itself has an overall green appearance when applied to skin as a result of light reflection from the pigment platelets. Preferred interference pigment materials for use in the composition of the invention have TiO₂ layer thicknesses of about 150 nm and about 250 nm, preferably about 150 nm. Suitable examples are those supplied by Merck under the trade name Timiron® especially Timiron® Silk Green, or supplied by Mearl under the trade name Flamenco®, especially Flamenco® Satin Green.

Precise levels of the pigment required in order to give the desired colour shift depend to some extent upon the particular type of pigment used. The green pigment is generally present in compositions of the present invention at a level of from about 0.1% to about 0.5%, preferably from about 0.15% to about 0.4%, more preferably from about 0.2% to about 0.3%.

In the second aspect of the present invention the compositions compnse a green platelet-type interference pigment, having a TiO₂ layer thickness of from 120 nm to 160 nm or a whole number multiple thereof, in an amount of from 0.1% to 0.5% by weight of the composition.

Overall particle sizes for the green pigments are preferably within the range from about 0.1 to about 100 μm, more preferably from about 5 to about 50 μm for the largest dimension and preferably from about 100 to about 1500 nm in thickness.

Other pigments, including differently coloured pigments, can also be used within the present compositions provided the overall colour shifts are within the ambits cited.

Optional Components

The topical compositions of the present invention may comprise a wide variety of optional components, provided that such optional components are physically and chemically compatible with the essential components described herein, and do not unduly impair stability, efficacy or other use benefits associated with the compositions of the present invention. Optional components may be dispersed, dissolved or the like in the carrier of the present compositions.

Optional components include emollients, oil absorbents, antimicrobial agents, binders, buffering agents, denaturants, cosmetic astringents, external analgesics, film formers, humectants, opacifying agents, perfumes, pigments, skin soothing and healing agents, preservatives, propellants, skin penetration enhancers, solvents, suspending agents, emulsifiers, cleansing agents, thickening agents, solubilising agents, waxes, sunscreens, sunless tanning agents, antioxidants and/or radical scavengers, chelating agents, anti-acne agents, anti-inflammatory agents, desquamation agents/exfoliants, organic hydroxy acids, vitamins and natural extracts. Nonexclusive examples of such materials are described in Harry's Cosmeticology, 7th Ed., Harry & Wilkinson (Hill Publishers, London 1982); in Pharmaceutical Dosage Forms-Disperse Systems; Lieberman, Rieger & Banker, Vols. 1 (1988) & 2 (1989); Marcel Decker, Inc.; in The Chemistry and Manufacture of Cosmetics, 2nd. Ed., deNavarre (Van Nostrand 1962-1965); and in The Handbook of Cosmetic Science and Technology 1st Ed., Knowlton & Pearce (Elsevier 1993). can also be used in the present invention.

1. Organic Particulate Material

The compositions of the present invention preferably comprise an organic particulate material having a refractive index of from about 1.3 to about 1.7, the particulate material being dispersed in the composition and having a volume average particle size in the range of from about 5 to about 30 μm, preferably from about 8 to about 25 μm. Without wishing to be bound by theory, it is believed that the organic particulate material, being of at least equal diameter to the oil layer on the skin created by the compositions of the present invention, acts as a non-greasy lubricant which improves the overall skin feel. The volume average particle size is measured when the particulate material is in the neat form i.e. in the essentially pure, powder form prior to combination with the carrier of the invention. Particular methods of measuring particle size may, however, require the particulate material to be dispersed in an inert carrier, such as a pure oil, in order to measure the particle size distribution. Particle size can be determined by any suitable method known in the art, such as by using coulter-counter equipment or the ASTM Designation E20-85 “Standard Practice for Particle Size Analysis of Particulate Substances in the Range of 0.2 to 75 Micrometers by Optical Microscopy”, ASTM Volume 14.02, 1993.

Refractive index can be determined by conventional methods. For example, a method for determining the refractive index, applicable to the present invention, is described in J. A. Dean, Ed., Lange's Handbook of Chemistry, 14th Ed., McGraw Hill, N.Y., 1992, Section 9, Refractometry. The refractive index is preferably in the range from about 1.35 to about 1.6, this range closely matching the refractive index of skin.

The compositions of the present invention preferably comprise from about 0.1% to about 10%, more preferably from about 0.3% to about 5%, especially from about 0.5% to about 2%, of the organic particulate material.

Preferred particulates are free-flowing, porous, materials, especially those having spheroidal particles. Suitable organic particulate materials include those made of polymethylsilsquioxane, referenced above, polyamide, polythene, polyacrylonitrile, polyarylic acid, polymethacrylic acid, polystyrene, polytetrafluoroethylene (PTFE) and poly (vinylidene chloride). Copolymers derived from monomers of the aforementioned materials can also be used. Preferred are polyamides, especially nylon. Particularly preferred for use herein are porous, nylon particles having a volume average particle size in the range of from about 5 to about 25 μm. Suitable nylon particles are commercially available from Elf Atochem SA, Paris, France under the tradename Orgasol®.

The compositions may contain other inorganic or organic particulate materials. However, it is preferred that the organic particulates in the compositions of the invention consist essentially of the particulate material described in this section entitled “Organic Particulate Material.”

2. Inorganic Matting Agent

Another optional component of the compositions of the present invention is an inorganic matting agent such as titanium or zinc oxides. However, when present, the matting agent is used at a level of no more than 3% to avoid undesirable skin whitening or an unnaturally ‘opaque’ appearance. Preferred for use herein is titanium dioxide and especially anatase titanium dioxide.

Anatase titanium oxide has a density of about 3.90 g/cm³ and a tetragonal, cubic close packed structure. The refractive index of anatase titanium oxide is 2.55. Anatase titanium dioxide is available from Kobo Products Inc. under the trade name Kobo BTD 11S2, from Whittaker, Clark, Daniels, South Plainfield, N.J., USA, under the trade name TiO₂ 9729, and from Cardre Inc., South Plainfield, N.J., USA, under the trade name Carde 70429.

The preferred matting agents for use herein from the viewpoint of skin feel, skin appearance and emulsion compatibility are coated pigments. The pigments can be treated with compounds such as amino acids such as lysine, silicones, lauroyl, collagen, polyethylene, lecithin and ester oils. The most preferred matting agents are the organosilicon (polysiloxane) treated pigments, for example polysiloxane treated titanium dioxide. Most preferred is polysiloxane treated anatase titanium dioxide.

A highly preferred matting agent is one which has been coated with an organosilicon component selected from a polyorganosiloxane or a silane wherein the coated pigment has a hydrogen potential of less than about 2.0, preferably less than about 1.0, more preferably less than about 0.5 ml, and especially less than about 0.1 ml H₂/g of coated pigment. The pigment is incorporated into the oil phase in the compositions herein. The coatings used can be bonded to the pigment surface by covalent bonding, physical adsorption or adhesion, preferably by covalent bonding to the surface of the pigment. The function of the coatings herein is hydrophobically to modify the pigments so that they are “wettable” in an oil phase of oil-in-water emulsions.

The total concentration of inorganic matting agent may be from about 0% to about 3% and, if used, preferably from about 0.1% to about 2.5%, more preferably from about 0.25% to about 2%.

3. Active for Chronically Regulating Skin Condition

In highly preferred embodiments, the compositions of the invention comprise a safe and effective amount of an active for chronically regulating skin condition selected from Vitamin B₃ compounds, retinoids, and combinations thereof. The aforementioned compounds may, when used by themselves, give rise to a sticky feel, especially when used at the higher levels. It has been found, however, that this sticky feel can be offset by using the organic particulates of the present invention. The compositions of the present invention preferably comprise from about 0.1% to about 15%, more preferably from about 0.3% to about 10%, even more preferably from about 1 to about 5% of the active. Specific examples of these actives include the following.

A. Vitamin B₃ Compounds

As used herein, “vitamin B₃ compound” means a compound having the formula:

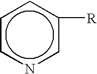

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. As used herein, “non-vasodilating” means that the ester does not commonly yield a visible flushing response after application to the skin in the subject compositions (the majority of the general population would not experience a visible flushing response, although such compounds may cause vasodilation not visible to the naked eye, i.e., the ester is non-rubefacient). Non-vasodilating esters of nicotinic acid include tocopherol nicotinate and inositol hexanicotinate; tocopherol nicotinate is preferred.

Other derivatives of the vitamin B₃ compound are derivatives of niacinamide resulting from substitution of one or more of the amide group hydrogens. Examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid (C₈H₈N₂O₃) and nicotinyl hydroxamic acid (C₆H₆N₂O₂). Exemplary nicotinyl alcohol esters include nicotinyl alcohol esters of the carboxylic acids salicylic acid, acetic acid, glycolic acid, and palmitic acid and the like. Other examples of vitamin B₃ compounds useful herein are 2-chloronicotinamide, 6-methylnicotinamide, N-methyl-nicotinamide, and niaprazine.

Examples of the above vitamin B₃ compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

One or more vitamin B₃ compounds may be used herein. Preferred vitamin B₃ compounds are niacinamide and tocopherol nicotinate. Niacinamide is more preferred.

Salts of the vitamin B₃ compound are also useful herein. Useful examples include organic or inorganic salts, such as inorganic salts with anionic inorganic species (e.g. chloride), and organic carboxylic acid salts. These and other salts of the vitamin B₃ compound can be readily prepared by the skilled artisan, for example, as described by W. Wenner, “The Reaction of L-Ascorbic and D-Isoascorbic Acid with Nicotinic Acid and Its Amide”, J. Organic Chemistry, VOL. 14, 22-26 (1949). Wenner describes the synthesis of the ascorbic acid salt of niacinamide.

In preferred embodiments, the ring nitrogen of the vitamin B₃ compound is uncomplexed, or after delivery to the skin becomes uncompleted. More preferably, the vitamin B₃ compound is essentially uncomplexed. Therefore, if the composition contains the vitamin B₃ compound in a salt or otherwise complexed form, such complex is preferably substantially reversible upon delivery of the composition to the skin Such complex should be substantially reversible at a pH of from about 5.0 to about 6.0. Such reversibility can be readily determined by one having ordinary skill in the art. In a preferred embodiment, the vitamin B₃ compound typically contains less than about 50% of the compound in a salt form.

The vitamin B₃ compound may be included as the substantially pure material, or as an extract obtained by suitable physical or chemical isolation from natural (e.g., plant) sources. The vitamin B₃ compound is preferably substantially pure i.e. substantially free of impurities arising from the original source. Substantially pure compounds can be provided in solution, optionally with an anti-oxidant or other stabiliser.

B. Retinoids

As used herein, “retinoid” includes all natural and/or synthetic analogues of Vitamin A or retinol-like compounds which possess the biological activity of Vitamin A in the skin as well as the geometric isomers and stereoisomers of these compounds. The retinoid is preferably retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid) or its esters such as tocopheryl retinoate. Preferably, retinoids other than retinoic acid are used. These compounds are well known in the art and are commercially available from a number of sources, e.g., Sigma Chemical Company (St. Louis, Mo.), and Boerhinger Mannheim (Indianapolis, Ind.). Other retinoids which are useful herein are described in U.S. Pat. Nos. 4,677,120, issued Jun. 30, 1987 to Parish et al.; 4,885,311, issued Dec. 5, 1989 to Parish et al.; 5,049,584, issued Sep. 17, 1991 to Purcell et al.; 5,124,356, issued Jun. 23, 1992 to Purcell et al.; and Reissue 34,075, issued Sep. 22, 1992 to Purcell et al. Preferred retinoids are the retinol esters such as retinyl palmitate, retinyl acetate, and retinyl propionate. Most preferred are retinyl propionate and retinyl palmitate.

The retinoid may be included as the substantially pure material, or as an extract obtained by suitable physical and/or chemical isolation from natural (e.g., plant) sources. The retinoid is preferably substantially pure.

The compositions of this invention optionally contain a safe and effective amount of the retinoid, such that the resultant composition is safe and effective for regulating skin condition, preferably for regulating visible and/or tactile discontinuities in skin, more preferably for regulating signs of skin ageing, even more preferably for regulating visible and/or tactile discontinuities in skin texture associated with skin ageing. The compositions preferably contain from about 0.005% to about 2%, more preferably 0.01% to about 2%, retinoid. Retinol is most preferably used in an amount of from or about 0.01% to about 0.15%; retinol esters are most preferably used in an amount of from about 0.01% to about 2% (e.g., about 1%); retinoic acids are most preferably used in an amount of from about 0.01% to about 0.25%; tocopheryl retinoate is preferably used in an amount of from about 0.01% to about 2%. The compositions herein can comprise both a retinoid and a Vitamin B₃ compound.

4. Emollients

The topical compositions of the subject invention generally comprise from about 1% to about 50%, preferably from about 3% to about 15% of a dermatologically acceptable emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics, Science and Technology, 2nd Edition, Vol. 1, pp. 32-43 (1972) contains numerous examples of materials suitable as an emollient. Illustrative examples of emollients include:

i) Straight and branched chain hydrocarbons having from about 7 to about 40 carbon atoms, such as dodecane, squalane, cholesterol, hydrogenated polyisobutylene, isohexadecane and the C₇-C₄₀ isoparaffins, which are C₇-C₄₀ branched hydro-carbons.

ii) C₁-C₃₀ alcohol esters of C₁-C₃₀ carboxylic acids and of C₂-C₃₀ dicarboxylic acids, e.g. isononyl isononanoate, isopropyl myristate, myristyl propionate, isopropyl stearate, behenyl behenate, dioctyl maleate, diisopropyl adipate, and diisopropyl dilinoleate.

iii) mono-, di- and tri-glycerides of C₁-C₃₀ carboxylic acids and ethoxylated derivatives thereof, e.g., caprylic/capric triglyceride, PEG-6 caprylic/capric triglyceride.

iv) alkylene glycol esters of C₁-C₃₀ carboxylic acids, e.g. ethylene glycol mono- and di-esters, and propylene glycol mono- and di-esters of C₁-C₃₀ carboxylic acids e.g., ethylene glycol distearate.

v) C₁-C₃₀ mono- and poly-esters of sugars and related materials. These esters are derived from a sugar or polyol moiety and one or more carboxylic acid moieties. Depending on the constituent acid and sugar, these esters can be in either liquid or solid form at room temperature. Examples include: glucose tetraoleate, the galactose tetraesters of oleic acid, the sorbitol tetraoleate, sucrose tetraoleate, sucrose pentaoleate, sucrose hexaoleate, sucrose heptaoleate, sucrose octaoleate, sorbitol hexaester in which the carboxylic acid ester moieties are palmitoleate and arachidate in a 1:2 molar ratio, and the octaester of sucrose wherein the esterifying carboxylic acid moieties are laurate, linoleate and behenate in a 1:3:4 molar ratio. Other materials include cottonseed oil or soybean oil fatty acid esters of sucrose. Other examples of such materials are described in WO 96/16636, incorporated by reference herein. A particularly preferred material is known by the INCI name sucrose polycottonseedate.

vi) Organopolysiloxane oils. The organopolysiloxane oil may be volatile, non-volatile, or a mixture of volatile and non-volatile silicones. The term “non-volatile” as used in this context refers to those silicones that are liquid under ambient conditions and have a flash point (under one atmospheric of pressure) of or greater than about 100° C. The term “volatile” as used in this context refers to all other silicone oils. Suitable organopolysiloxanes can be selected from a wide variety of silicones spanning a broad range of volatilities and viscosities. Non-volatile polysiloxanes are preferred. Suitable silicones are disclosed in U.S. Pat. No. 5,069,897, issued Dec. 3, 1991. Preferred for use herein are organopolysiloxanes selected from the group consisting of polyalkylsiloxanes, alkyl substituted dimethicones, dimethiconols, polyalkylaryl siloxanes, and mixtures thereof More preferred for use herein are polyalkylsiloxanes and cyclomethicones. Preferred among the polyalkylsiloxanes are dimethicones.

vii) Vegetable oils and hydrogenated vegetable oils. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, partially and fully hydrogenated oils from the foregoing sources, and mixtures thereof.

viii) animal fats and oils, e.g. cod liver oil, lanolin and derivatives thereof such as acetylated lanolin and isopropyl lanolate. Lanolin oil is preferred.

ix) Also useful are C₄-C₂₀ alkyl ethers of polypropylene glycols, C₁-C₂₀ carboxylic acid esters of polypropylene glycols, and di-C₈-C₃₀ alkyl ethers, examples of which include PPG-14 butyl ether, PPG-15 stearyl ether, dioctyl ether, dodecyl octyl ether, and mixtures thereof.

Highly preferred compositions of the invention comprise, as emollients, from about 2% to about 20%, preferably from about 3% to about 10%, more preferably from about 3% to about 8%, of a high spreading oil having a contact angle of less than about 40°. These high spreading oils are useful for distributing vitamin B₃ compounds on skin and for providing the right balance between lubricity and spreadability to the entire emollient system. Preferably the relative weights of the entire emollient system are selected such that the entire oil phase has a contact angle of less than about 40°, preferably less than about 35° and more preferably less than about 30°. Preferred high spreading oils are selected from:

i) branched chain hydrocarbons having a weight average molecular weight of from about 100 to about 1000 and

ii) liquid ester emollients of formula I:

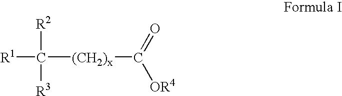

wherein R¹ is selected from H or CH₃, R², R³ and R⁴ are independently selected from C₁-C₂₀ straight chain or branched chain alkyl, and x is an integer of from 1 to 20.

Suitable examples of branched chain hydrocarbons include isododecane, isohexadecane and isoeicosane. Preferred is isohexadecane. The poly(alphaolefin) anti-tack agents herein, described in further detail below, are typically also branched chain hydrocarbons. When these anti-tack agents are used then their amount is to be considered included in the levels of high spreading oils referred to above.

Suitable ester emollient materials of Formula I above include methyl isostearate, isopropyl isostearate, isostearyl neopentanoate. isononyl isononanoate, isodecyl octanoate, isodecyl isononanoate, tridecyl isononanoate, myristyl octanoate, octyl pelargonate, octyl isononanoate, myristyl myristate, myristyl neopentanoate, isostearyl neopentanoate, myristyl octanoate, myristyl propionate, isopropyl myristate and mixtures thereof Preferred ester emollients for use herein are isononyl isononanoate, isostearyl neopentanoate, methyl isostearate, isopropyl isostearate, isopropyl stearate, isopropyl myristate and mixtures thereof.

Particularly preferred high spreading oils for use herein are isohexadecane, isononyl isononanoate, methyl isostearate, isopropyl isostearate, or mixtures thereof.

5. Anti-Tack Agents

Particularly when elevated levels of glycerin or vitamin B₃ compounds, especially niacinamide, are used in the compositions of the invention, the inclusion of from about 0.3% to about 4%, preferably from about 0.5% to about 2.5%, more preferably from about 1% to about 2%, of an anti-tack agent can be beneficial. Preferred anti-tack agents are selected from a polyalphaolefins having a MW of from about 260 to about 1000 and occlusive agents selected from petrolatum, cetyl ricinoleate and lanolin. Whilst not completely understood it appears that the polyalphaolefin anti-tack agents and the occlusive anti-tack agents act via different mechanisms. Nevertheless, both are effective in reducing the sticky skin feel associated with elevated levels of glycerine or vitamin B₃ compounds. Although mixtures of the anti-tack agents are not excluded, best benefits are obtained when the anti-tack agent is selected from just one of the two classes.

Suitable polyalphaolefins as described above can be derived from 1-alkene monomers having from about 6 to about 14 carbon atoms, preferably from about 6 to about 12 carbon atoms, especially from about 8 to about 12 carbon atoms. The polyalphaolefins useful herein are preferably hydrogenated polyalphaolefin oligomers. Examples of 1-alkene, monomers for use in preparing the polyalphaolefin oligomers herein include 1-hexane, 1-octene, 1-decene, 1-dodecene, 1-tetradecene, branched chain isomers such as 4-methyl-1-pentene, and combinations thereof Most preferred are oligomers of 1-octene to 1-dodecene or combinations thereof. Preferred is polydecene. Suitable polydecenes are commercially available from Mobil Chemical Company, P.O. Box 3140, Edison, N.J. 08818, USA, under the tradename Puresyn® 4 and from BP Amoco of 200 E. Randolph Drive, Chicago, Ill. 60601-7125 under the tradename Silkflo® 364.

Most preferred as an anti-tack agent is petrolatum.

6. Humectants

A highly preferred optional component is a humectant, particularly of the polyhydric alcohol-type. Typical polyhydric alcohols include polyalkylene glycols and more preferably alkylene polyols and their derivatives, including propylene glycol, dipropylene glycol, polypropylene glycol, polyethylene glycol and derivatives thereof, sorbitol, hydro-xypropyl, sorbitol, erythritol, threitol, pentaerythritol, xylitol, glucitol, mannitol, hexylene glycol, butylene glycol (e.g., 1,3-butylene glycol), hexane triol (e.g., 1,2,6-hexanetriol), glycerine, ethoxylated glycerine and propoxylated glycerine.

Also useful herein are sodium 2-pyrrolidone-5-carboxylate, guanidine; glycolic acid and glycolate salts (e.g. ammonium and quaternary alkyl ammonium); lactic acid and lactate salts (e.g. ammonium and quatemary alkyl ammonium); aloe Vera in any of its variety of forms (e.g., aloe vera gel); hyaluronic acid and derivatives thereof (e.g., salt derivatives such as sodium hyaluronate); lactamide monoethanolamine; acetamide monoethanolamine; urea; panthenol; sodium pyroglutamate (NaPCA), water-soluble glyceryl poly(meth)acrylate lubricants (such as Hispagel®) and mixtures thereof.

The above listed compounds may be incorporated singly or in combination. Preferred humectants are selected from glycerine, glyceryl polyacrylate, urea, panthenol and mixtures thereof.

7. Emulsifiers/Surfactants

Compositions herein preferably contain an emulsifier and/or surfactant, generally to help disperse and suspend the discontinuous phase within the continuous phase. A surfactant may also be useful if the product is intended for skin cleansing. For convenience hereinafter emulsifiers will be referred to under the term ‘surfactants’, thus ‘surfactant(s)’ will be used to refer to surface active agents whether used as emulsifiers or for other surfactant purposes such as skin cleansing. Known or conventional surfactants can be used in the composition, provided that the selected agent is chemically and physically compatible with essential components of the composition, and provides the desired characteristics. Suitable surfactants include silicone materials, non-silicone materials, and mixtures thereof.

The compositions of the present invention preferably comprise from about 0.05% to about 15% of a surfactant or mixture of surfactants. The exact surfactant or surfactant mixture chosen will depend upon the pH of the composition and the other components present.

Preferred surfactants are nonionic. Among the nonionic surfactants that are useful herein are those that can be broadly defined as condensation products of long chain alcohols, e.g. C₈₋₃₀ alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C₈₋₃₀ alkyl group. Examples of long chain alcohols from which the alkyl group can be derived include decyl alcohol, cetyl alcohol, stearyl alcohol, lauryl alcohol, myristyl alcohol, oleyl alcohol and the like. Preferred examples of these surfactants include those wherein S is a glucose moiety, R is a C₈₋₂₀ alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel).

Other useful nonionic surfactants include the condensation products of alkylene oxides with fatty acids (i.e. alkylene oxide esters of fatty acids). These materials have the general formula RCO(X)ₙOH wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C₁₀₋₃₀ alkyl group, X is —OCH₂CH₂— (i.e. derived from ethylene glycol or oxide) or —OCH₂CHCH₃— (i.e. derived from propylene glycol or oxide), and n is an integer from about 6 to about 100 and R′ is H or a C10-30 alkyl group. Still other nonionic surfactants are the condensation products of alkylene oxides with both fatty acids and fatty alcohols [i.e. wherein the polyalkylene oxide portion is esterified on one end with a fatty acid and etherified (i.e. connected via an ether linkage) on the other end with a fatty alcohol]. These materials have the general formula RCO(X)ₙOR′ wherein R and R′ are C₁₀₋₃₀ alkyl groups, X is —OCH₂CH₂ (i.e. derived from ethylene glycol or oxide) or —OCH₂CHCH₃— (derived from propylene glycol or oxide), and n is an integer from about 6 to about 100, examples of which include ceteth-6, ceteth-10, ceteth-12, ceteareth-6, ceteareth-10, ceteareth-12, steareth-6, steareth-10, steareth-12, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amide surfactants, which are described in more detail in WO 98/04241.

Preferred among the nonionic surfactants are those selected from the group consisting of steareth-2, steareth-21, ceteareth-20, ceteareth-12, sucrose cocoate, steareth-100, PEG-100 stearate, and mixtures thereof.

Other nonionic surfactants suitable for use herein include sugar esters and polyesters, alkoxylated sugar esters and polyesters, C₁-C₃₀ fatty acid esters of C₁-C₃₀ fatty alcohols, alkoxylated derivatives of C₁-C₃₀ fatty acid esters of C₁-C₃₀ fatty alcohols, alkoxylated ethers of C1-C30 fatty alcohols, polyglyceryl esters of C₁-C₃₀ fatty acids, C₁-C₃₀ esters of polyols, C₁-C₃₀ ethers of polyols, alkyl phosphates, polyoxyalkylene fatty ether phosphates, fatty acid amides, acyl lactylates, and mixtures thereof. Examples of these non-silicon-containing surfactants include: polysorbate 20, polyethylene glycol 5 soya sterol, steareth-20, ceteareth-20, PPG-2 methyl glucose ether distearate, ceteth-10, poly-sorbate 80, polysorbate 60, glyceryl stearate, sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

Another emulsifier useful herein are fatty acid ester blends based on a mixture of sorbitan or sorbitol fatty acid ester and sucrose fatty acid ester, the fatty acid in each instance being preferably C₈-C₂₄, more preferably C₁₀-C₂₀. The preferred fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the trade name Arlatone 2121.

The hydrophilic surfactants useful herein can alternatively or additionally include any of a wide variety of cationic, anionic, zwitterionic, and amphoteric surfactants such as are known in the art. See, e.g., McCutcheon's, Detergents and Emulsifiers, North American Edition (1986), published by Allured Publishing Corporation; and U.S. Pat. No. 5,011,681 to Ciotti et al., issued Apr. 30, 1991.

Preferred emulsions of the present invention include a silicone containing emulsifier or surfactant. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C₂-C₃₀ pendant side chains. Still other useful dimethicone copolyols include materials having various cationic, anionic, amphoteric, and zwitterionic pendant moieties.

8. Thickening Agent (Including Thickeners and Gelling Agents)

The compositions of the present invention can also comprise a thickening agent, generally from about 0.1% to about 5% and preferably from about 0.25% to about 2%.

Suitable thickening agents include cellulose and derivatives such as cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof.

Other useful thickeners include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof Also useful are acrylic acid/ethyl acrylate copolymers and the carboxyvinyl polymers sold by the B.F. Goodrich Company under the trade mark of Carbopol resins. Suitable Carbopol resins are described in WO98/22085.

Preferred compositions of the present invention include a thickening agent selected from carboxylic acid polymers, crosslinked polyacrylates, polyacrylamides, xanthan gum and mixtures thereof more preferably selected from polyacrylamide polymers, xanthan gum and mixtures thereof Preferred polyacrylamides are predispersed in a water-immiscible solvent such as mineral oil and the like, containing a surfactant (HLB from about 7 to about 10) which helps to facilitate water dispersibility of the polyacrylamide. Most preferred for use herein is the non-ionic polymer under the CTFA designation: polyacrylamide and isoparaffin and laureth-7, available under the trade name Sepigel 305 from Seppic Corporation.

9. Anti-Inflammatory Agents

A safe and effective amount of an anti-inflammatory agent may be added to the compositions of the subject invention, preferably from about 0.1% to about 5%, more preferably from about 0.1% to about 2%, of the composition. The anti-inflammatory agent enhances the skin appearance benefits of the present invention, e.g., such agents contribute to a more uniform and acceptable skin tone or colour. The exact amount of anti-inflammatory agent to be used in the compositions will depend on the particular anti-inflammatory agent utilised since such agents vary widely in potency.

Anti-inflammatory agents useful herein include steroids such as hydrocortisone; non-steroidal anti-inflammatory drugs (NSAIDS) such as ibuprofen; panthenol and ether and ester derivatives thereof e.g. panthenol ethyl ether, panthenyl triacetate; pantothenic acid and salt and ester derivatives thereof especially calcium pantothenate; aloe vera, bisabolol, allantoin and compounds of the liquorice (the plant genus/species Glycyrrhiza glabra) family, including glycyrrhetic acid, glycyrrhizic acid, and derivatives thereof e.g. salts such as ammonium glycyrrhizinate and esters such as stearyl glycyrrhetinate.

10. Sunscreens and Sunblocks.

Compositions of the subject invention can contain a sunscreen. Suitable sunscreens can be organic or inorganic. Especially preferred organic sunscreens include butylmethoxy-dibenzoylmethane, 2-ethylhexyl-p-methoxycinnamate, phenyl benzimidazole sulfonic acid, and octocrylene. Inorganic sunscreens include zinc oxide and titanium dioxide. Exact amounts used will vary depending upon the sunscreen chosen and the desired Sun Protection Factor (SPF) but inorganic sunscreens are preferably used only at low levels as described above for matting agents. An agent may also be added to any of the compositions useful in the subject invention to improve the skin substantivity of those compositions, particularly to enhance their resistance to being washed off by water, or rubbed off. A preferred agent which will provide this benefit is a copolymer of ethylene and acrylic acid. Compositions comprising this copolymer are disclosed in U.S. Pat. No. 4,663,157, Brock, issued May 5, 1987.

11. Anti-Oxidants/Radical Scavengers

Compositions of the subject invention can further include an anti-oxidant/radical scavenger. The anti-oxidant/radical scavenger is especially useful for providing protection against UV radiation which can cause increased scaling or texture changes in the stratum corneum and against other environmental agents which can cause skin damage. Suitable amounts are from about 0.1% to about 10%, more preferably from about 1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate), β-carotene, tocopherol (vitamin E), tocopherol sorbate, tocopherol acetate, other esters of tocopherol, butylated hydroxy benzoic acids and their salts, gallic acid and its alkyl esters, especially propyl gallate, uric acid and its salts and alkyl esters, sorbic acid and its salts, amines (e.g., N,N-diethylhydroxylamine, amino-guanidine), sulfhydryl compounds (e.g., glutathione), dihydroxy fumaric acid and its salts, bioflavonoids, lysine, methionine, proline, superoxide dismutase, silymarin, tea extracts, grape skin/seed extracts, melanin, and rosemary extracts may be used. Preferred anti-oxidants/radical scavengers are selected from tocopherol acetate, tocopherol sorbate and other esters of tocopherol, more preferably tocopherol acetate.

12. Chelants

The inclusion of a chelating agent or chelant is especially useful for providing protection against UV radiation which can contribute to excessive scaling or skin texture changes and against other environmental agents which can cause skin damage. A suitable amount is from about 0.01% to about 1%, preferably from about 0.05% to about 0.5%, of the composition. Exemplary chelants that are useful herein are disclosed in U.S. Pat. No. 5,487,884. Preferred chelants are ethylenediamine tetraacetic acid (EDTA), furildioxime and derivatives thereof.

13. Desquamation Agents/Exfoliants

A safe and effective amount of a desquamation agent may be added to the compositions of the subject invention, more preferably from about 0.1% to about 10%, even more preferably from about 0.2% to about 5%, also preferably from about 0.5% to about 4% of the composition. Desquamation agents enhance the skin appearance benefits of the present invention. For example, the desquamation agents tend to improve the texture of the skin (e.g., smoothness). A variety of desquamation agents are known in the art and are suitable for use herein, including organic hydroxy acids such as salicylic acid, glycolic acid, lactic acid, 5-octanoyl salicylic acid, hydroxyoctanoic acid, hydroxycaprylic acid, and lanolin fatty acids. One desquamation system that is suitable for use herein comprises sulphydryl compounds and zwitterionic surfactants and is described in WO 96/01101. Another suitable desquamation system comprises salicylic acid and zwitterionic surfactants and is described in WO 95/13048. Salicylic acid is preferred.

14. Skin Lightening Agents

The compositions of the present invention can also comprise a skin lightening agent. When used, the compositions preferably comprise from about 0.1% to about 10%, more preferably from about 0.2% to about 5%, also preferably from about 0.5% to about 2%, of a skin lightening agent. Suitable skin lightening agents include those known in the art, including kojic acid, arbutin, ascorbic acid and derivatives thereof, e.g., magnesium ascorbyl phosphate. Further skin lightening agents suitable for use herein also include those described in WO 95/34280 and WO 95/23780.

Preparation of Compositions

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like.

Methods of Use

The compositions of the present invention are useful for providing a more even skin tone, especially to the hands or other non-facial parts of the body.

A wide range of quantities of the compositions of the present invention can be employed to provide a skin appearance and/or feel benefit. Quantities of the present compositions which are typically applied per application are, in mg composition/cm² skin, from about 0.1 mg/cm² to about 10 mg/cm². A particularly useful application amount is about 2 mg/cm². Typically applications would be on the order of about once per day, however application rates can vary from about once per week up to about three times per day or more.

The compositions of this invention provide a visible improvement in skin condition essentially immediately following application of the composition to the skin. Such immediate improvement involves coverage or masking of skin imperfections such as textural discontinuities (including those associated with skin ageing, such as enlarged pores), and/or providing a more even skin tone or colour.

Compositions of the invention which comprise an active for chronically regulating skin also provide visible improvements in skin condition following chronic topical application of the composition. “Chronic topical application” and the like involves continued topical application of the composition over an extended period during the subject's lifetime, preferably for a period of at least about one week, more preferably for a period of at least about one month, even more preferably for at least about three months, even more preferably for at least about six months, and more preferably still for at least about one year. Chronic regulation of skin condition involves improvement of skin condition following multiple topical applications of the composition to the skin. Typically applications would be on the order of about once per day over such extended periods, however application rates can vary from about once per week up to about three times per day or more.

### EXAMPLES

The following examples further describe and demonstrate embodiments within the scope of the present invention. They are given for the purpose of illustration and are not to be construed as limitations of the present invention. Where applicable, ingredients are given in CTFA name. All of the examples are oil-in-water emulsions prepared using conventional formulating techniques.

| | Example | | |
|---|---|---|---|
| | 1 | 2 | 3 |
| Ingredient | % w/w | % w/w | % w/w |
| Niacinamide | 2.0 | 5.0 | 6.0 |
| Retinyl propionate | — | — | 0.2 |
| Nylon-12¹ | 2.0 | 3.0 | 0.5 |
| Titanium Dioxide (and) Mica²-green | 0.25 | 0.1 | 0.5 |
| Titanium Dioxide (and) Mica³-white | 0.75 | 1.5 | 0.5 |
| Polyacrylamide & isoparaffin & laureth-7 | 2.25 | 2.0 | 1.0 |
| Xanthan gum | — | — | 0.3 |
| Titanium dioxide⁴ | — | — | 0.3 |
| Glycerine | 7.0 | 5.0 | 3.0 |
| Urea | — | — | 2.0 |
| Panthenol | 1.0 | 0.5 | 3.0 |
| Salicylic acid | — | 1.5 | — |
| Allantoin | 0.2 | 0.1 | 0.05 |
| Aloe vera gel | 0.01 | — | 0.05 |
| Tocopheryl acetate | 0.5 | — | 0.05 |
| Cetyl alcohol | 2.0 | 1.0 | 1.25 |
| Stearyl alcohol | 2.0 | 1.0 | 1.25 |
| Cyclomethicone & dimethiconol | 0.75 | 1.5 | 0.50 |
| Steareth-21 | 0.6 | 0.4 | 0.3 |
| Steareth-2 | 0.1 | 0.08 | 0.03 |
| Sorbitan stearate & sucrose cocoate | 1.5 | — | — |
| Stearyl alcohol | 0.5 | 1.0 | 1.5 |
| Cetyl alcohol | 0.5 | 1.0 | 1.5 |
| Behenyl alcohol | 0.5 | 1.0 | 1.5 |
| Isohexadecane | 3.0 | 5.0 | 7.0 |
| Petrolatum | 0.5 | 1.0 | 2.0 |
| PPG-15 stearyl ether | 3.0 | 5.0 | 4.00 |
| Sucrose polycottonseedate | — | 3.0 | — |
| Dimethicone (350 mm²s⁻¹) | 0.5 | — | 1.0 |
| Disodium EDTA | 0.02 | 0.01 | 0.05 |
| Ethyl paraben | 0.1 | 0.15 | 0.2 |
| Propyl paraben | 0.1 | 0.15 | 0.2 |
| Methyl paraben | 0.1 | 0.15 | 0.2 |
| Benzyl Alcohol | 0.5 | 0.2 | 1.0 |
| Phenoxetol | 0.5 | — | 1.0 |
| Perfume | qs. | qs. | qs. |
| De-ionised water | to 100% | to 100% | to 100% |

¹Orgasol ® 2002 EXD NAT COS.
²A green interference pigment.
³An optically neutral titanium dioxide coated mica.
⁴Silicone coated anatase form.

On application to a subject's hands, at the rate of 2 mg /cm² skin, an essentially immediate visual improvement in skin appearance is provided e.g., reduced visibility of pores and a more even skin tone. Continuation of application at the same rate once or twice daily for a period of 3-6 months improves skin surface texture, including diminishing fine lines and wrinkles, in addition to the essentially immediate improvements in appearance.

1. A topical, liquid, cosmetic composition comprising:
a) from 0.3% to 1.5% of an optically neutral phyllosilicate; and
b) a green pigment in an amount effective to give the composition a Δa colour shift value of from −1 to −5 and a Δb colour shift value of from −2 to −5, when applied to the skin or a skin substitute at a coverage of 2.4 mg cm^{-2.},
wherein the total level of inorganic particles in the composition is less than 5%.

2. The composition of claim 1 wherein the optically neutral phyllosilicate is a mica.

3. The composition of claim 1 wherein the green pigment is a platelet-type interference pigment, having a TiO₂ layer thickness of from 120 nm to 160 nm or a whole number multiple thereof, in an amount of from 0.1% to 0.5% by weight of the composition.

4. A topical liquid, cosmetic composition comprising:
a) from 0.3% to 1.5% of an optically neutral phyllosilicate; and
b) a green platelet-type interference pigment, having a TiO₂ layer thickness of from 120 nm to 160 nm or a whole number multiple thereof, in an amount of from 0.1% to 0.5% by weight of the composition;
wherein the total level of inorganic particles in the composition is less than 5%.

5. The composition of any preceding claim which comprises an oil phase having a contact angle of less than about 40°.

6. The composition of any preceding claim which is an oil in water emulsion.

7. The composition of any preceding claim which further comprise from 0.1% to 10% of a organic particulate material having a refractive index of from 1.3 to 1.7, the particulate material being dispersed in the composition and having a volume average particle size in the range of from 5 to 30 μm.

8. A method of providing a more even skin tone comprising topically applying the composition of any preceding claim.

9. A method according to claim 8 wherein the composition is applied to the hands.
